(19) United States
(12) Patent Application Publication
(10) Pub. No.: US 2024/0085390 A1
(43) Pub. Date: Mar. 14, 2024

(54) COMPACT SCENT DELIVERY DEVICE AND COMPACT SCENT DELIVERY SYSTEM

(71) Applicant: Firmenich SA, Satigny (CH)

(72) Inventors: Denis SHCHERBAKOV, Plainsboro, NJ (US); Artem KIRSHON, Plainsboro, NJ (US); Addi FADEL, Plainsboro, NJ (US); Nicholas O'LEARY, Plainsboro, NJ (US); Ralf INAUEN, Schlieren (CH); Ronny HOFFMANN, Schlieren (CH); Manuel WEBER, Schlieren (CH); Branislav BALAZ, Schlieren (CH); Alessandro CASILLI, Plainsboro, NJ (US); Stefan SIEBER, Schlieren (CH)

(21) Appl. No.: 18/263,048

(22) PCT Filed: Feb. 4, 2022
(86) PCT No.: PCT/EP2022/052718

§ 371 Date: Jul. 26, 2023

Related U.S. Application Data
(60) Provisional application No. 63/145,813, filed on Feb. 4, 2021.

(30) Foreign Application Priority Data
May 10, 2021 (EP) 21200978.1

(51) Int. Cl. G01N 33/00

(52) U.S. Cl. CPC G01N 33/0001; G01N 33/0004

(57) ABSTRACT

The compact scent delivery system (300) comprises: —a compact scent delivery device (100, 200), —an air flow mixing unit (400) comprising: 5—an inlet (405) for scented air, configured to be connected to an outlet (125) of scented air of the compact scent delivery device, —an outlet (410) for mixed scented air directed at a smelling port (415), —a conduit (420) connecting the inlet for scented air and the outlet for mixed diluted scent air and 0—at least one element of: —an inlet (425) for fresh air, configured to be connected to a fresh air delivery apparatus, said inlet delivering fresh air into the scented air stream between the inlet for scented air and the outlet for mixed scented air and/or 5—an outlet (430) for waste scented air, configured to be connected to an air flow retrieval apparatus and/or a waste air exhaust, said outlet retrieving scented air from the scented air stream between the inlet for scented air and the outlet for mixed scented air to reduce the air flow either for subsequent dilution or for the 0 associated smelling port (415) or both.

### TECHNICAL FIELD OF THE INVENTION

The present invention relates to a compact scent delivery device and to a compact scent delivery system. It applies, in particular, to the fields of olfactometry, perfumery, fine fragrance perfumery, fragrance design, and flavor design.

### BACKGROUND OF THE INVENTION

Sensory performance evaluations are an integral part of developing fragranced or flavored products, such as but not limited to air fresheners, scented laundry detergents, scented or flavored powders, hair care products, fine fragrances, surface cleaners, and candles.

In perfumery, sensory evaluations are typically conducted with a panel of human subjects, recording sensory intensity and other characteristics of a fragrance released from either a “free oil” (the fragrance concentrate) or a substrate that represents consumer product category for which the fragrance is being developed. For fine fragrances, examples of such substrates may include but are not limited to paper blotters, glass slides and skin. For laundry detergents, the substrate of interest may be a towel that has been washed with a scented detergent. For hair care products, sensory evaluations may be conducted using hair as the substrate, washed with a scented shampoo and/or conditioner. For air fresheners, the substrates may include but are not limited to paper products, a wood-based or a wood-like materials, gels, woven or non-woven fabrics, and polymer-based products such as plastics.

Such evaluations may be conducted in large ventilated spaces, such as cabins or rooms of approximately two cubic meters in volume or larger, whereby a fragrance source, such as a fragranced product or substrate or free oil, is placed somewhere inside the cabin, allowed to ventilate in the cabin at a prescribed air flow or air change rate (ventilation rate) for a prescribed amount of time to fragrance the air of the cabin, and then evaluated by sniffing through a window in the cabin door or by entering the cabin space.

There are a number of limitations and disadvantages associated with using cabins and rooms for sensory evaluations. First, the size and number of such dedicated facilities needed for side-by-side product development and evaluations require substantial floor space and construction investment. Second, such facilities are not transportable and do not allow flexibility of location for the sensory evaluations. Finally, due to large, fixed dimensions and limited ventilation capabilities in terms of available air ventilation rates, such facilities allow a limited set of conditions for conducting sensory evaluations, particularly concerning the extent of fragrance dilution in the air, which is problematic for development and testing of consumer fragrance products in numerous applications.

In particular, evaluation of fragrance trail (also known as “sillage” in French) for fine fragrances is linked to the technical problems discussed above. Trail performance is a key attribute and selling point of fine fragrances that is demanded by consumers worldwide. A robust, flexible, and differentiating evaluation method for trail performance of fine fragrances is essential for development of superior consumer products, but the need for a device with flexible settings for air flow dilution, compact footprint, and transportable format that can generate real human fragrance trail for evaluation still remains unmet in the fragrance industry. Fragrance trail being a particular example, this need is not limited to trail and includes also other categories of fragranced products where evaluation of fragrance performance under real conditions of consumer use is necessary for development of superior products.

In the particular example of fragrance trail, attempts have been made in the prior art to meet this need, but they have been inadequate because the underlying engineering design principles of the devices for assessment of fragrance trail do not provide accurate representation of real human trail. For example, various air convection prototypes have been proposed to provide distance between the fragrance source and point of evaluation via tubes or channels, attempting to duplicate the distance between the fragrance wearer and the evaluator; however, when fresh air is delivered over a fragrance source via a rotating fan, the scented air quickly becomes well-mixed and the dependence of air dilution on distance within such device is thereby greatly reduced or completely removed, making it unsuitable for simulating a full range of conditions associated with human fragrance trail. Other attempts to introduce the concept of sillage evaluation were limited by the sheer size of the fragrance emitting device and the open design of the down-stream scented air channel, making the device not transportable and unable to allow precise control over the extent of gas-phase dilution of the fragrance being evaluated. These attempts suffer from the limitations mentioned above: size and footprint of the evaluation method, lack of reproducibility, and particularly inability to replicate the real-life fragrance trail conditions.

Unlike many other scent performance attributes, trail is associated with movement. This can be either the movement of a person wearing fragrance (walking) or the movement of the air around the person (wind or ventilation) or both (person walking in the street, exposed to natural wind currents). Movement induces a certain degree of air convection, transporting the fragrance away from the source (the person wearing fragrance) and allowing other people to smell the fragrance. Trail performance of fine fragrances is generally judged by the maximum distance from the source at which the fragrance remains at least perceptible, and preferably recognizable, to other people. It is important to note that, whenever significant gas-phase concentration gradients exist, such as with surrounding unscented air, the extent of fragrance dilution in the air is correlated strongly with distance from the fragrance source at which the evaluation is performed.

Currently, there exists no satisfactory system in the prior art allowing for a realistic, fast, reproducible, compact, and transportable solution for sensory evaluation of fragrance performance that would be suitable to multiple consumer product categories, air fresheners, cleaning products, laundry products, candles, reeds, and body care products. Furthermore, there exists no satisfactory system allowing for a realistic, fast, reproducible, compact, and transportable solution for sensory evaluation of the trail performance of a fragrance, such as a fine fragrance.

Previous systems such as disclosed in the document US 20190128782 are known. This document discloses a device and associated method for evaluating specifically “sillage” (trail) performance attribute of fragrances. The device is essentially a long, straight tube with several smelling ports located at different distances from the fragrance source. At one end of the tube, a fragrance sample is placed in front of a fan that blows fresh air over the fragrance sample at approximately 0.7 m/s. Fragranced air is then transported from the source by convective flow from the fan and evaluated at one or multiple smelling ports along the length of the tube.

Such a system requires much space, does not provide much flexibility of operating conditions, and the accuracy of the reproduction of the trail phenomenon is low. In particular, as the rotational air flow from a fan quickly mixes the fragrance into the air and no additional fresh air is introduced between the fragrance source and the smelling ports, the fragrance headspace transported in the tube becomes distance-invariant after a very short distance from the source upstream of the first smelling port, meaning that the fragrance concentration in the air delivered at subsequent downstream ports of the device is essentially the same after the device reaches steady-state operating mode.

Previous systems such as disclosed in the document U.S. Pat. No. 6,067,842 are also known. This document discloses an olfactometer comprising a sniffing port, a sample supply for generating a sample flow constituted by a carrier gas and sample head space from a saturation chamber and for supplying the sample flow to the sniffing port, a carrier gas supply, and mixer for predetermined dilution of the sample flow. The carrier gas supply includes mass flow controllers with variable flow rates disposed before the saturation chamber for providing variable carrier gas flow to the saturation chamber. A multiplicity of capillaries of different diameter connects the mixing means with the sniffing port via individual injectors. A computer may be interfaced with the olfactometer to regulate the amount of sample and/or carrier gas in the sample flow.

Such systems, however, while being optimized for olfactometry of small odorant samples, present major shortcomings for the purpose of evaluating fragrance performance of consumer products in real conditions of product use. The most obvious shortcoming is the design of the saturation chamber, being unable to accommodate most full-size fragranced consumer products to be placed inside the unit for generation of scented air. The second shortcoming is how the fragrance source is subjected to air flow (ventilation) inside the saturation chamber, lacking the flexibility of design and operating parameters to expose the fragranced product inside the chamber to varying degrees of direct air convection, the latter being important for controlling kinetics of evaporation and aging of the fragranced product.

Previous systems such as disclosed in the document US 20090320559 are also known. Such systems aim at an apparatus for evaluating the fragrance characteristics of a substance comprises a cylindrical chamber in which is located a block for supporting a substance, e.g., a fragrance to be evaluated. A flow of air, generated by a fan, passes through the chamber, around the substance and leaves the chamber via a main outlet and a plurality of sensing outlets each provided with a metering valve leading the air flow to a corresponding nasal mask. Individuals take two or three sniffs of the air flow from the mask and record their findings on a pre-prepared form.

Such systems also offer multiple shortcomings: (1) inlet air flow generated by a fan introduces rotational flow into the chamber that increases air mixing inside the scent generation chamber, diminishing the functionality of the air flow shield placed inside the chamber; (2) no auxiliary dilution of the scented air exiting the main chamber is included in the design, which significantly reduces ability of the device to mimic real conditions of use of fragranced products and makes it impossible to separately control evaporation kinetics of said products and their scent intensity presented to human panelists.

### SUMMARY OF THE INVENTION

The present invention is intended to remedy all or part of these disadvantages.

To this effect, according to a first aspect, the present invention aims at compact scent delivery device, comprising:
a dynamic air flow chamber, comprising:
   an inlet for fresh air,
   a fragrance source support,
   an outlet for scented air and
the fragrance source support, fragrance source support being located within the chamber between the inlet and the outlet along a stream of air traversing the chamber.

Such provisions allow for the use of a dynamic system, subject to continuous air flow, thus providing scented air as a function of the features of the air flow at the inlet (in addition to the features of the fragranced product placed inside the device).

In a particular embodiment, the chamber comprises:
the inlet for fresh air, said inlet facing a reflection surface configured to disperse and reflect the air towards the fragrance source support,
said reflection surface,
the fragrance source support, which can be perforated in certain embodiments of this invention, being located within the chamber between the inlet and the outlet along a stream of air traversing the chamber,
the outlet for scented air and
a narrowing air flow guide located between the fragrance source support and the outlet along a stream of air traversing the chamber.

These provisions allow circumventing the above-mentioned limitations by providing a scaled down device intended as a replacement for test cabins typically used in the fragrance industry for assessing fragrance performance for various consumer product formats.

Despite a small footprint, the device object of the present invention can accommodate full-sized consumer products and reproduce olfactive performance observed in sensory cabins, rooms, and other consumer end-use environments. The device can be designed with adjustable operational parameters that can be adapted to changing business needs, client requirements, and different geographical locales.

Furthermore, these provisions:
increase bandwidth of sensory tests and bring sensory testing capability to evaluators where full-size test cabins are not suitable,
help improve fragrance design by incorporating better, more rigorous evaluation conditions and methods early in the development process (such as different size rooms, different distances from the fragrance source, etc),
provide superior capability to demonstrate product performance and
provide hygienic evaluation of cross category fragrances (skin, fragrance on hair, deposition on fabric, etc.).

In particular embodiments, the fragrance source support comprises an air flow guide.

Such embodiments allow for the accurate reproduction of the influence a larger air volume would have on an equivalent fragrance sample positioned in the open air.

In particular embodiments, the fragrance source support comprises a perforated surface, perforations of the surface forming the air flow guide.

Such embodiments allow for the two-fold advantage of providing a fragrance support as well as an air flow guide in a single item.

In particular embodiments, the perforated surface (that also serves as the fragrance support) spans the cross-section of the chamber.

Such embodiments force the air flow to enter the perforations, thus providing additional control over the air flow profiles within the chamber.

In particular embodiments, the device object of the present invention comprises a perforation adaptation means configured to close/open at least one perforation of the surface.

Such embodiments allow for the dynamic adjustment of the operation parameters of the device, such as evaporation kinetics of the fragrance product placed inside the chamber.

In particular embodiments, an opening of the air inlet located within the chamber is located at a distance from the reflection surface less than or equal to three times the width of said opening.

Such embodiments allow for the optimal circulation of air within the chamber.

In particular embodiments, the inlet extends longitudinally within the chamber, said inlet comprising a fixture for the fragrance source support.

According to a second aspect, the present invention aims at a compact scent delivery system, comprising:
a compact scent delivery device object of the present invention,
an air flow mixing unit comprising:
an inlet for scented air, configured to be connected to the outlet of scented air of the compact scent delivery device,
an outlet for mixed scented air directed at a smelling port,
a conduit connecting the inlet for scented air and the outlet for mixed scent air and
at least one element of:
   an inlet for fresh air, configured to be connected to a fresh air delivery apparatus, said inlet delivering fresh air into the scented air stream between in the inlet for scented air and the outlet for mixed scented air and/or
   an outlet for waste scented air, configured to be connected to an air flow retrieval apparatus, said inlet retrieving scented air from the scented air stream between in the inlet for scented air and the outlet for mixed scented air to reduce the air flow.

These provisions provide similar advantages to the compact scent delivery device object of the present invention, as well additional improvements allowing for the dynamic adjustment of the air flow provided to the smelling port and additional control over the scent concentration (intensity) delivered to the smelling port. This allows, even with a standard vial, to simulate how consumers perceive fragrance at different distances from the fragrance source by adjusting air flow rate at the inlet of fresh air, outlet flow rate of waste scented air and auxiliary dilutions with additional fresh downstream of the scent delivery device.

In particular embodiments, the conduit comprises at least one elbow or flow modification means to enhance air mixing.

Such embodiments allow for the improvement of mixing within the air flow.

In particular embodiments, the system object of the present invention comprises at least two elements among:
an inlet for fresh air, configured to be connected to a fresh air delivery apparatus, said inlet delivering fresh air into the scented air stream between the inlet for scented air and the outlet for mixed scented air and/or
an outlet for waste scented air, configured to be connected to an air flow retrieval apparatus, said inlet retrieving scented air from the scented air stream between in the inlet for scented air from the compact scent delivery device and the outlet for mixed scented air to the smelling/evaluation port(s),
   in which the elbow or other flow modification means to enhance air mixing is located between two said elements.

Such embodiments allow for the size, noise and air flow pump demand reduction, allowing for a larger dynamic dilution range in a small space.

In particular embodiments, the conduit comprises a needle or any other geometric obstacle in the air flow path configured to increase mixing within the conduit.

Such embodiments allow for the improvement of mixing within the air flow conduit.

In particular embodiments, at least one inlet for fresh air or outlet for waste scented air is oriented perpendicularly to the airstream with which at least one said inlet for fresh air or outlet for waste scented air interacts.

Such embodiments allow for the creation of minor turbulences or flow disturbances within the airstream.

In particular embodiments, the system object of the present invention comprises a heating apparatus, said heating apparatus being configured to air flow from the inlet to the outlet.

Such embodiments provide an ease of cleaning of the conduits of the system.

In particular embodiments, the system object of the present invention comprises an air flow restrictor upstream of the outlet for mixed scented air or the smelling/evaluation port.

Such embodiments allow for the additional regulation of the air flow velocity.

In particular embodiments, the system object of the present invention comprises at least one pump or compressed air source connected to an inlet for fresh air or an outlet for wasted scented air.

In particular embodiments, the system object of the present invention comprises an electronic command unit comprising:
a dilution and/or waste ratio determination means and
at least one mass flow controller connecting at least one said pump or compressed air source to an inlet for fresh air or an outlet for wasted scented air and
a command means configured to transmit activation commands to at least one said mass flow controller as a function of the dilution and/or waste ratio determined.

Such embodiments allow for the dynamic activation and control of the system.

In particular embodiments, the command unit further comprises a scent trail simulation means, activation commands being transmitted by the command means as a function of parameters of the simulated scent trail.

Such embodiments allow for the simulation of scent trail in terms of air flow and dilution in order to match user perception of real-life trail conditions.

In particular embodiments, the system object of the present invention comprises a virtual reality headset or an electronic display, the dilution and/or waste ratio being determined as a function of an execution parameter of said virtual reality headset or electronic display.

Such embodiments allow for the matching of the scent dilution delivered by the scent delivery system to a virtual distance between the user and a virtual fragrance source in a virtual environment.

In particular embodiments, the air dilution ratio executed by the scent delivery system decreases or increases over time.

In particular embodiments, the system object of the present invention comprises at least two compact scent delivery devices object of the present invention and, for each compact scent delivery device a mixing unit, said system further comprising a scented air mixer connected to the outlet for mixed scented air of each said mixing unit, said scented air mixer further comprising an outlet for mixed scented air directed at a smelling port.

Such embodiments allow for the mixing of fragrances.

In particular embodiments, a compact scent delivery device is positioned so that the scented air outlet is higher than the inlet for fresh air of said device.

In particular embodiments, the system object of the present invention comprises at least two compact scent delivery devices, said system further comprising a fragrance source selector configured to alternatively connect the inlet for scented air with one compact scent delivery device.

In particular embodiments, the system object of the present invention comprises a moisture trapping means and/or an activated carbon trapping means positioned along the stream of air traversing the system.

Such embodiments allow for the optimal operation of the system, avoiding deactivation due to the presence of unwanted moisture or chemical compounds within the system.

According to a third aspect, the present invention aims at an assembly to form a system object of the second aspect of the present invention, comprising:
a compact scent delivery device according to the first aspect of the present invention,
an air flow mixing unit comprising:
   an inlet for scented air, configured to be connected to the outlet of scented air of the compact scent delivery device,
   an outlet for mixed scented air directed at a smelling port,
   a conduit connecting the inlet for scented air and the outlet for mixed scented air and
   at least one element of:
      an inlet for fresh air, configured to be connected to a fresh air delivery apparatus, said inlet delivering fresh air into the scented air stream between in the inlet for scented air and the outlet for mixed scented air and/or
      an outlet for waste scented air, configured to be connected to an air flow retrieval apparatus, said inlet retrieving scented air from the scented air stream between in the inlet for scented air and the outlet for mixed scented air to reduce the air flow at the smelling port.

The advantages of this aspect are similar to the aspects previously disclosed above.

According to a fourth aspect, the present invention aims at a method of scent delivery comprising:
a step of injecting air into a compact scent delivery device according to the first aspect of the present invention,
a step of injecting the scented air, from the outlet of the compact scent delivery device, into an air flow mixing unit and
a step of providing fresh air into the scented air stream in the mixing unit and/or a step of retrieving waste scented air from the mixing unit.

Such an aspect presents the same advantages as the system object of the present invention.

In particular embodiments, the method object of the present invention comprises a step of determining a scented air dilution ratio, at least one step of providing and/or retrieving being operated as a function of the ratio determined.

Such an aspect presents the same advantages as the system object of the present invention.

In particular embodiments, the ratio determined during the step of determining is determined as a function of a simulation parameter of distance between a scent source and a location of perception of said scent, said distance influencing intensity, perceivability and/or recognizability of the scented air at the outlet of the scent delivery device.

Such an aspect presents the same advantages as the system object of the present invention.

### BRIEF DESCRIPTION OF THE DRAWINGS

Other advantages, purposes and particular characteristics of the invention shall be apparent from the following non-exhaustive description of at least one particular device or system which is the object of this invention, in relation to the drawings annexed hereto, in which:

FIG. 1 represents, schematically, a first embodiment of a device object of the present invention,

FIG. 2 represents, schematically, a second embodiment of a device object of the present invention,

FIG. 3 represents, schematically, a first view of a first particular embodiment of a system object of the present invention,

FIG. 4 represents, schematically, a second view of a first particular embodiment of a system object of the present invention,

FIG. 5 represents, schematically, a third view of a first particular embodiment of a system object of the present invention,

FIG. 6 represents, schematically, a succession of steps of a particular embodiment of the method object of the present invention,

FIG. 7 represents, schematically, a view of a second particular embodiment of a system object of the present invention,

FIG. 8 represents, schematically, a view of a third particular embodiment of a system object of the present invention and

FIG. 9 represents, schematically, a view of a particular embodiment of a scented air flow circuit object of the present invention.

### DETAILED DESCRIPTION OF THE INVENTION

This description is not exhaustive, as each feature of one embodiment may be combined with any other feature of any other embodiment in an advantageous manner.

It should be noted at this point that the figures are not to scale.

The terms “fresh air” are intended as lacking at least any predetermined fragrance. Preferably such fresh air is intended as lacking any type of fragrance to provide the user with a more realistic experience of the fragrance housed within a compact scent delivery device 100. Such fresh air can be ambient air, for example, or air presenting a predetermined composition and located within a storage unit accessible to the device 100.

The terms “fragrance source” can designate any kind of fragrant ingredient or combination of ingredients, such as air freshener products, various substrates treated with fragrance (paper, tile, towels, hair etc.), powders, liquids, and/or candles for example.

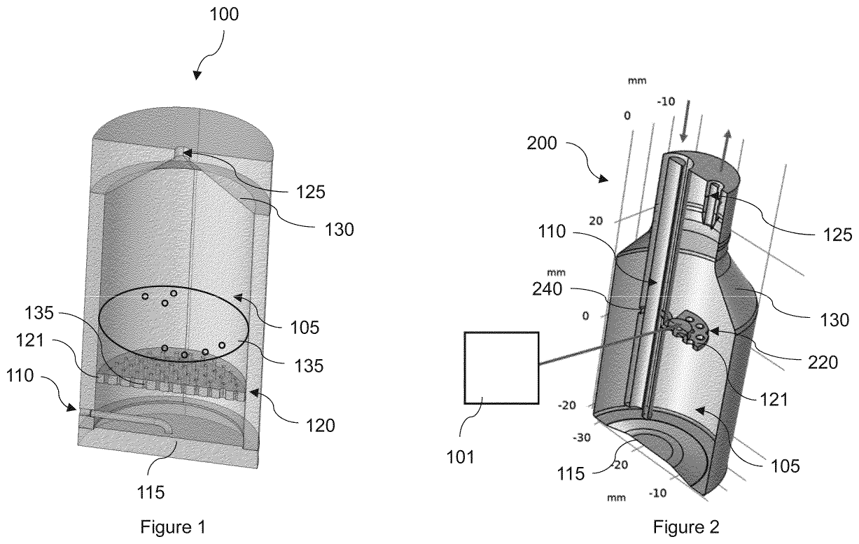

Both FIGS. 1 and 2 show a particular embodiment of the device, 100 and 200, object of the present invention. This compact scent delivery device, 100 or 200, comprises:
a dynamic air flow chamber 105, comprising:
   an inlet 110 for fresh air, said air being preferably dried (via a moisture capturing means 355 for example) and filtered of contaminants (via an activated carbon 356 filter for example),
   a fragrance source support, 120 or 220,
   an outlet 125 for scented air and
the fragrance source support, 120 or 220, fragrance source support being located within the chamber between the inlet and the outlet along a stream of air traversing the chamber.

The chamber 105 is configured in such a way to not be at equilibrium. In other words, it is configured to be under continuous ventilation, which allows for the creation of a dynamic headspace chamber.

FIG. 1 shows a particular embodiment of the device 100 object of the present invention. In this compact scent delivery device 100, the chamber 105 comprises:
the inlet 110 for fresh air, said inlet facing a reflection surface 115 configured to disperse and reflect the air towards the fragrance source support, 120 or 220,
said reflection surface,
the outlet 125 for scented air and
a narrowing air flow guide 130 located between the fragrance source support and the outlet along a stream of air traversing the chamber.

The chamber 105 can be defined as a volume of a predetermined size enclosed within a shell. The shell may be formed of any material that is preferably not porous to the chemical ingredients traversing the chamber 105. Said shell is preferably formed in a rigid material presenting no capacity for expansion at the operating pressures intended for the device 100, thus forcing the air flow at a constant pressure imposed by the pressure of the inlet 110 for fresh air.

The chamber 105 can be formed of several, interconnectable, parts. In a particular example, the chamber 105 is formed of two parts joined at a cross-section of the chamber 105.

The inlet 110 for fresh air can be formed by an opening in the chamber 105. This inlet 110 can be associated with any type of temporary or permanent fixture configured to fit a conduit to transport fresh air to the inlet 110. This conduit can, in turn, be connected to a fresh air providing means, such as a pump or a compressed air storage for example.

The inlet 110 provides an opening preferably near the reflection surface 115 of the device 100. In such variants, the distance from the opening of the inlet 110 to the reflection surface 115 can correspond to less than 20%, or less than 10%, of the height of the chamber 105. The height of the chamber is defined by the general axis, such as the cylindrical axis of symmetry of the chamber where such geometric symmetry is present, which the air flow follows within the chamber 105. Preferably, the device 100 is configured to be positioned vertically, the opening of the inlet 110 facing downwards and the outlet 125 being positioned above said opening such that the height of the chamber 105 can generally be defined as an axis parallel to the axis defined by the opening of the inlet 110 to the outlet 125, or an equivalent opening thereof.

Preferably, an opening of the inlet 110 located within the chamber 105 is located at a distance from the reflection surface 115 less than or equal to three times the diameter of said opening.

Forcing the air flow to flow vertically, that is along a vector comprising at least one component opposite to the force of gravity, allows for the scented air to be ventilated uniformly and consistently while avoiding accumulation.

Preferably, the device 100 is configured to be positioned vertically, with the reflection surface 115 being at the base of the device 100.

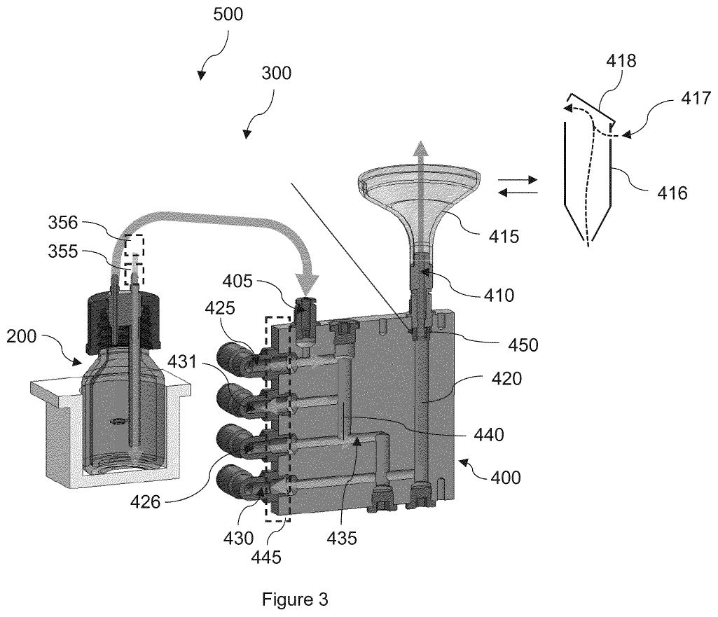

Similarly, the outlet 125 for scented air can be formed by an opening in the chamber 105. This outlet 125 can be associated with any type of temporary or permanent fixture configured to fit a conduit to the scented fresh air from the outlet 125 to a smelling port or to a mixing unit 400 such as represented in FIGS. 3 to 5.

Within the chamber, the circulation of the air flow is optimized to provide an air flow presenting similar fragrance transport and dilution parameters to the context of a fragrance source located within a large volume of air, which corresponds to the intended use case for perception of the fragrance.

In order to optimize the air flow traversing the chamber 105, this chamber 105 comprises a reflection surface 115 towards which the air flow entering the inlet 110 is directed.

This reflection surface 115 can be a part of the shell surrounding the inner volume of the chamber 105 or a dedicated surface fixed within the chamber. The purpose of this reflection surface 115 is to diffuse (disperse, distribute, spread out) the air flow entering the chamber 105 throughout the cross-section of the chamber. The fragrance source is thus indirectly touched by the air flow entering the chamber 105.

The reflection surface 115 may be formed in such a way as to direct the air flow towards a predetermined direction within the chamber 105. Such a variant is represented in FIG. 2, wherein the reflection surface 115 is convex in shape. Reflection surface can also be comprised of concave curvature or a combination of convex and concave curvatures.

To further optimize the circulation of the air flow within the chamber 105, said chamber 105 comprises a narrowing air flow guide 130. The term “narrowing”, in this context, is not limited to a circular or elliptical or conical cross-section. This air flow guide 130 preferably presents a diminishing cross-section surface along the air flow stream, regardless of the shape of the cross-section. In other words, an upper section of the chamber 105 may be tapered.

The chamber 105 is designed to shape an air flow circulation in the proximity of a fragrance source. This fragrance source is supported by a fragrance source support 120, which can take a variety of shapes insofar as the fragrance remains upon the support 120 when the device 100 is stationary and in operating conditions.

In a variant, such as represented in FIG. 1, the support 120 spans the entire cross-section of the chamber 105. In another variant, such as represented in FIG. 2, the support 220 only spans part of the cross-section of the chamber 105.

In particular embodiments, such as represented in FIGS. 1 and 2, the support, 120 and 220, comprises a perforated surface 121. In such embodiments, the perforations 135 may span the entire cross-section of the chamber that is generally perpendicular to the overall direction of the air flow (cross-sectional width of the surface 121), said perforations 135 behaving similarly to tubes in which the air is forced to flow. In such variants, the perforations form an air flow guide 135. In variants, the perforations only span part of the width of the surface 121.

In particular embodiments, the perforated surface 121 spans the entire cross-section of the chamber 105.

In particular embodiments, the device 100 object of the present invention comprises a perforation adaptation means 135 configured to close/open at least one perforation of the surface.

Said perforation adaptation means 135 can be, in simple embodiments, a surface configured to block all or part of the perforations of the perforated surface 121. In more complex embodiments, at least one perforation can be dynamically opened or closed as a function of an activation/deactivation command received. Such a dynamic opening/closing can be obtained, for example, via a mechanical actuator such as used in microfluidic systems. Such an actuator can be obtained via microvalves feeding a fluid to an inflatable pocket configured to expand and block a perforation upon activation of the corresponding microvalve.

The fragranced product, or source, can be elevated from the bottom of the device, 100 or 200, and placed on a height-adjustable stand/pedestal, which could be the same as the aforementioned perforated support, which lifts away from the bottom of the device, 100 or 200, so that the flow profile is well-established and consistent before reaching the fragrance source. Depending on an embodiment, the pedestal/stand can contain an array of holes for air permeation.

Preferably, the support, 120 or 220, is located prior to the narrowing air flow guide 130 along the stream of air traversing the chamber 105.

In particular embodiments, the support, 120 or 220, is located in the bottom third of the height of the chamber 105.

FIG. 2 shows a particular embodiment of the device 200 object of the present invention. In this compact scent delivery device 200, the chamber 105 comprises:
the inlet 110 for fresh air, said inlet facing a reflection surface 115 configured to disperse and reflect the air towards the fragrance source support, 220,
said reflection surface,
the outlet 125 for scented air,
the fragrance source support 220, fragrance source support being located within the chamber and between the inlet and the outlet along a stream of air traversing the chamber, and
a narrowing air flow guide 130 located between the fragrance source support and the outlet along a stream of air traversing the chamber.

In this embodiment, the inlet 110 extends longitudinally within the chamber 105, said inlet comprising a fixture 240 for the fragrance source support. Such an embodiment allows for a more precise air flow stream design.

The fixture 240 is, for example, a ridge positioned on the external surface of the inlet 110, within the chamber 105, said ridge being configured to associate with a corresponding annular grip of the fragrance source support 220.

FIG. 2 shows, a particular embodiment of the device 200 object of the present invention. This device 200 comprises a micro compounder 101 or any similar liquid-phase mixer, configured to mix a fragrance from its individual ingredients in liquid phase and deliver the liquid fragrance to a substrate, such as a robotic pipette dispensing fragrance upon the fragrance support.

In particular embodiments, such as shown in FIG. 3, the device 200 comprises a moisture trapping means 355, such as a condenser, positioned along the stream of air traversing the system.

In particular embodiments, such as shown in FIG. 3, the device 200 comprises an odor trapping means 356, such as an activated carbon filter or cartridge, positioned along the stream of air traversing the system.

FIG. 3 shows a particular embodiment of the system 300 object of the present invention. This compact scent delivery system 300 comprises:
a compact scent delivery device, 100 or 200, corresponding to any variant of the device, 100 or 200, disclosed in regard to FIG. 1 or 2,
an air flow mixing unit 400 comprising:
   an inlet 405 for scented air, configured to be connected to the outlet 125 of scented air of the compact scent delivery device 200,
   an outlet 410 for mixed scented air directed at a smelling port 415 or 416,
   a conduit 420 connecting the inlet for scented air and the outlet for mixed scent air and
   at least one element of, and preferably at least one element of each of, the following:
      an inlet 425 for fresh air, configured to be connected to a fresh air delivery apparatus, said inlet delivering fresh air into the scented air stream between in the inlet for scented air and the outlet for mixed scented air and/or
      an outlet 430 for waste scented air, configured to be connected to an air flow retrieval apparatus, said inlet retrieving scented air from the scented air stream between in the inlet for scented air and the outlet for mixed scented air to adjust the air flow at the smelling port.

Preferably, the compact scent delivery device 200 is positioned in such a way that the scented air outlet 125 is higher along a vertical direction than the outlet 110 for fresh air of said device.

The inlet 405 for scented air can be formed by an opening of the conduit 420. This inlet 405 can be associated with any type of temporary or permanent fixture configured to fit a conduit to transport scented air from the outlet 125 of the device 200 to the inlet 405 of the mixing unit 400.

The outlet 410 for mixed scented air can be formed by an opening of the conduit 420. This outlet 410 can be associated with any type of temporary or permanent fixture configured to fit a smelling port 415 or 416 to receive the mixed scented air from the outlet 410 of the mixing unit 400.

Such a smelling port can be an olfactometry cone 415, a nasal cannula or any other customized shape. In a particular embodiment, the smelling port may comprise an elongated volume 416, preferentially positioned vertically, said volume comprising a vent hole 417 within the volume envelope and optionally a cap 418, allowing for redirecting the airflow toward the user.

In particular embodiments, at least one inlet 425 for fresh air or outlet 430 for waste scented air is oriented perpendicularly to the airstream with which at least one said inlet 425 for fresh air or said outlet 430 for waste scented air interacts.

The conduit 420 can be formed by drilling within a block of unitary material, such as steel for example. In variants, the conduit 420 is flexible, although such variants provide little design predictability in terms of the impact on the intended air flow and disadvantageous in terms of heating the unit for cleaning.

In other variants, the conduit 420 may be formed by machining the conduit in a block of material, such as an aluminum block for example, and covering said conduit by another complementary block of material, to be attached and sealed to the block in which the conduit was machined.

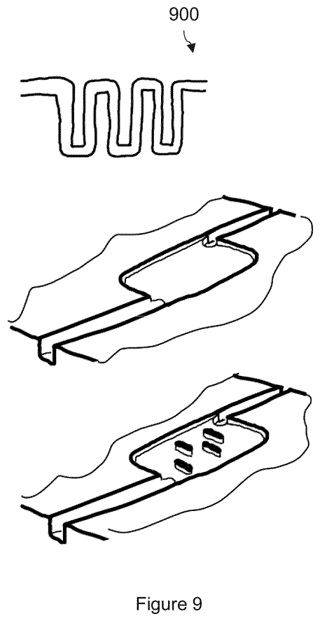

Such variants 900 are exemplified in FIG. 9.

The mixing unit 400 comprises at least one element of, and preferably at least one element of each of, the following:
an inlet 425 for fresh air, configured to be connected to a fresh air delivery apparatus, said inlet delivering fresh air into the scented air stream between in the inlet for scented air and the outlet for mixed scented air and/or
an outlet 430 for waste scented air, configured to be connected to an air flow retrieval apparatus and/or a waste air exhaust, said outlet retrieving scented air from the scented air stream between in the inlet for scented air and the outlet for mixed scented air, as a means to control serial dilutions of scented air and to adjust the air flow at the smelling port.

Such an inlet 425 for fresh air can be formed by an opening of the conduit 420. This inlet 425 can be associated with any type of temporary or permanent fixture configured to fit a conduit to transport fresh air from a source of fresh air to the inlet 425 of the mixing unit 400.

Preferably, the inlet 425 for fresh air is connected to a pump, or another source of fresh air, such a compressed air source, and a flow controller configured to inject a determined amount of fresh air into the inlet 425 to dilute the amount of fragrance within the air flow directed at the smelling port 415, thus reducing the concentration of fragrance within the air flow.

The outlet 430 for scented air can be formed by an opening of the conduit 420. This outlet 430 can be associated with any type of temporary or permanent fixture configured to fit a conduit to receive the scented air from the outlet 430 of the mixing unit 400.

Preferably, the outlet 430 for scented air is connected to a pump and a flow controller configured to extract scented air from the conduit 420 to control serial dilutions of scented air and to adjust the air flow at the smelling port 415. Preferably, the flow rate at the inlet 405 for scented air is maintained constant. Preferably, the flow rate at the outlet 410 for mixed scented air is maintained constant. Such flow rate at the outlet 410 may correspond to the flow rate at the inlet 405. In such variants, if the inlet 425 for fresh air is activated and a determined quantity, or flow rate, of fresh air injected within the stream originating from the device 200 (or, equivalently, the device 100) to impart a certain amount of dilution of scented air, the outlet 430 extracts the same quantity, or flow rate, of waste scented air in order to maintain the flow rate of the stream constant.

In other embodiments, the outlet 430 is connected to a waste scented air exhaust means.

In other embodiments, a flow rate at the outlet 410 is determined, either automatically, via a computing system for example, or manually, via a mechanical or logical user selection means, the activation of the inlet 425 and outlet 430 being determined as a function of the determined flow rate.

In particular embodiments, such as shown in FIG. 3, the conduit 420 comprises at least one elbow 435 or other flow modification means.

An elbow 435 is defined by a sudden change of air flow direction caused by an angle in the conduit 420. Such an angle can generally be of 90 degrees. Other values can be used as well, provided the amount of turbulence within the air flow is consistent with the intention of the design of the mixing unit 400. Such other values can be, for example, of 30, 45 or 60 degrees.

In particular embodiments, such as shown in FIG. 3, the system 300 comprises at least two elements among:
an inlet, 425 and 426, for fresh air, configured to be connected to a fresh air delivery apparatus, such as a pump or a compressed air source, said inlet delivering fresh air into the scented air stream between in the inlet 405 for scented air and the outlet 410 for mixed scented air and/or
an outlet, 430 and 431, for waste scented air, configured to be connected to an air flow retrieval apparatus, such as a pump, and/or a waste air exhaust said inlet retrieving scented air from the scented air stream between in the inlet 405 for scented air from the compact scent delivery device and the outlet 410 for mixed scented air to the smelling/evaluation ports.

Any combinations of any number of inlets, 425 and 426, and/or any number of outlets, 430 and 431 can be implemented.

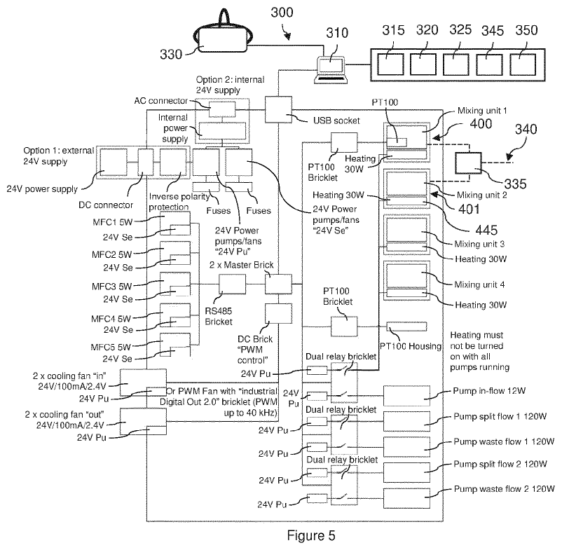

In particular embodiments, such as represented in FIG. 5, the system 300 further comprises a command unit 310 comprising:
a dilution and/or waste ratio determination means 315,
at least one mass flow controller 306 connecting at least one said pump 305 or compressed air source to an inlet, 425 and/or 426, for fresh air or to an outlet, 430 and/or 431, for waste, or discarded, scented air and
a command means 320 configured to transmit activation commands to at least one said mass flow controller as a function of the dilution and/or waste ratio determined.

The command unit 310 is, for example, a computer, tablet or phone. In broader terms, the command unit 310 may be a microcontroller configured to run a computer program upon a computing unit, said computer program acting as the dilution and/or waste ratio determination means 315.

The dilution and/or waste ratio determination means 315 is preferably configured to determine said ratio as a function of the air flow rate and/or gas-phase fragrance concentration at the outlet 125 of the scent delivery device and the air flow rate and/or a target gas-phase fragrance concentration to be achieved at the smelling port. The gas-phase fragrance concentration can be predetermined and stored in an electronic memory within the command unit 310 or obtained via a computer interface, such as a keyboard, mouse or touchscreen, from a user and stored in said electronic memory. In advanced embodiments, this gas-phase fragrance concentration is measured via a fragrance concentration sensor located between the outlet 125 of the device 200 and the inlet 405 of the mixing unit 400.

The target fragrance concentration can be set by a user, via a computer interface, or determined by the command unit 310. In such variants, an external parameter value can be responsible for the target fragrance concentration determined.

For example, if the target concentration is ten times lower than the concentration at the outlet 125 of the scent delivery device, the dilution ratio can be set to ten. This, in turn, means that the pump 305 associated to the air inlet, 425 or 426, and associated mass flow controller 306 are configured to inject ten times more fresh air than a pump or a compressed air source (not represented) providing fresh air to the fresh air inlet 110 of the device 200.

Preferably, the pump 305 provides a constant air flow associated to a mass flow controller 306 configured to use said air flow to provide fresh air to at least one inlet, 425 and/or 426, or to push the waste flow to at least one outlet, 430 and/or 431.

As the air flow pressure and velocity might be unsuitable for the user, a pump associated to the scented air outlet, 430 or 431, can remove the air flow with the same ratio as the pump 305 providing fresh air to the air inlet, 425 or 426. Ultimately, in this scenario, the air flow rate remains unchanged despite the change of concentration of the fragrance within said air flow.

A formula allowing for the determination of the dilution ratio might be:

$$Δ = \frac{{ρ}_{out}}{{ρ}_{in}}$$

Where:
Δ designates the dilution ratio,
ρᵢₙ in designates the fragrance gas phase concentration at the inlet 405 of the mixing unit 400 and
ρₒᵤₜ designates the fragrance gas phase concentration at the outlet 410 of the mixing unit 400,

A formula allowing for the determination of the activation of the inlet or inlets, 425 and/or 426, for fresh air might be:

Q_{Σinlet}=Δ·Qᵢₙ

Where:
Q_{Σinlet} designates the flow rate of the inlet or sum of flow rates of the inlets, 425 and/or 426, for fresh air and
Qᵢₙ designates the flow rate at the inlet 405 of the mixing unit 400.

A formula allowing for the determination of the activation of the outlet or outlets, 430 and/or 431, for waste scented air might be:

Q_{Σoutlet}=|Qₒᵤₜ−Q_{Σinlet}−Qᵢₙ|

Where:
Q_{Σoutlet} designates the flow rate of the outlet or sum of flow rates of the outlets, 430 and/or 431, for fresh air and
Qₒᵤₜ designates the flow rate at the outlet 410 of the mixing unit 400.

The total fragrance headspace dilution targeted for evaluation by the system 300 relates to specific product usage conditions of interest. For example, one scenario to be reproduced for evaluation with the system 300 could refer to an air freshener of a given size (commercial consumer product) being placed in a room of a specified size ventilated at two air changes per hour (typical product usage environment). Another scenario to be reproduced for evaluation with the system 300 is fragrance trail, where a person wearing fragrance on hair, skin, and/or clothing is walking and leaving behind a scent trail that is produced through movement and convective transport of fragrance in the surrounding air.

The flow rate desired at the smelling ports can be, for example, a minimum of 0.8 L/min, if fittings with reduced cross-sectional area such as nasal cannulas are used for presenting fragranced air to the nose of a user of the system 300. If an open olfactometry cone is used, then the desired flow rate range at the smelling ports is 2-20 L/min. These flow settings depend on the design of the cone, general guidance being to deliver fragranced air for presentation to an evaluator at speeds between 0.2 m/s and 1 m/s. In a portable/transportable embodiment of the system 300, the target flow rate at the smelling port(s) can be set preferably in the range of 0.8-3 L/min, with the dimensions of the smelling port chosen based on this target flow rate.

The air flow rate through the device, 100 or 200, is set based on the size/scale of the vessel used and the application targeted. In a portable embodiment of the system 300 for evaluation of fragrance trail, which utilizes device, 100 or 200, of approximately 30-mL inner cavity volume, a flow rate between 20 mL/min and 200 mL/min is chosen, corresponding to the range of 40 ACH (air changes per hour) to 400 ACH within the device, respectively. More optimally, the flow in the 30-mL device, 100 or 200, should be set in the range of 50 mL/min and 150 mL/min. In a non-portable embodiment of the system 300 with the more general-purpose evaluation scope (including full-size air fresheners, candles, fragrance trail, etc), which utilizes a scent release device, 100 or 200, of approximately 10-L volume, a flow rate between 0.3 L/min and 12 L/min is chosen, corresponding to the range of 1.8 ACH to 72 ACH within the device, respectively. More optimally, the flow in the 10-L device, 100 or 200, should be set in the range of 0.5 L/min and 7 L/min, corresponding to 3 ACH and 42 ACH, respectively.

A first stage of air dilution of the concentrated fragrance headspace, emanating from the fragrance source, takes place in the scent delivery device, 100 or 200. This first-stage dilution depends on the rate of fragrance release from the fragrance source, the flow rate of air through the device, 100 or 200, the geometry of the device, 100 or 200, the geometry of the fragrance source support and the orientation of the fragrance surface relative to the principal direction of air flow, although there may be other factors which are not listed. For describing fragrance headspace dilution in the device, 100 or 200, the headspace concentration at the outlet of the device, 100 or 200, is used in the denominator of the ratio defining dilution. Dilution ratio in the device, 100 or 200, consistent with the test set-up and operating conditions can be predicted from Computational Fluid Dynamics (CFD) or measured experimentally via analytical chemistry techniques, such as gas chromatography mass spectrometry, for example.

For a portable embodiment of the system 300 targeting reproduction of smelling fragrance at different distances from the source within a fragrance trail, the following is an example of different operating conditions that can be selected to achieve desired dilution between the outlet from the device, 100 or 200, and the smelling port(s), not accounting for the first-step dilution inside the device, 100 or 200:

| | | | | Split flow | | | | Split flow | | |
|---|---|---|---|---|---|---|---|---|---|---|
| | Fragrance | | Split flow | for a first | | Split flow | | for a first | | Fragrance |
| | inflow into | | for a first | outlet 430 | | for a second | | outlet 430 | | flow exiting |
| Ratio | the mixing | | inlet 425 | for waste | | inlet 426 | | for waste | | the mixing |
| of flows | unit 400 | | for fresh air | scented air | | for fresh air | | scented air | | unit 400 |
| 1:10 | 136 | mL/min | 1364 mL/min | 0 | mL/min | 0 | mL/min | 0 | mL/min | 1500 mL/min |
| 1:50 | 29 | mL/min | 1471 mL/min | 0 | mL/min | 0 | mL/min | 0 | mL/min | 1500 mL/min |
| 1:250 | 20 | mL/min | 4000 mL/min | 2819 | mL/min | 299 | mL/min | 0 | mL/min | 1500 mL/min |
| 1:500 | 20 | mL/min | 4000 mL/min | 3418 | mL/min | 898 | mL/min | 0 | mL/min | 1500 mL/min |
| 1:1000 | 20 | mL/min | 4000 mL/min | 3418 | mL/min | 1600 | mL/min | 502 | mL/min | 1500 mL/min |
| 1:1500 | 20 | mL/min | 4000 mL/min | 3418 | mL/min | 2600 | mL/min | 1502 | mL/min | 1500 mL/min |
| 1:2000 | 20 | mL/min | 4000 mL/min | 3418 | mL/min | 3600 | mL/min | 2502 | mL/min | 1500 mL/min |

The total dilution of the fragrance headspace generated by the device is the product of the first-step dilution inside the scent delivery device, 100 or 200, and the “Ratio of Flows” column of the table above, the latter describing operational settings for flow rate(s) in the dilution stream(s) (called “Split Flows”) and flow rate(s) in the waste stream(s) (called “Waste Flows”).

For example, if the device, 100 or 200, is operated at the dilution ratio of 40, and the total dilution to be achieved by the system 300 is 2000, then the dilution ratio in the intermediate steps (downstream of scent delivery device) should be 2000/40=50. This setting corresponds to row 2 of the table above for the flow rate of 1.5 L/min at the smelling port. The dilution ratio of 50 can also be achieved by other combinations of settings for the diluting flows and the waste flows, particularly if different flow rate at the smelling port(s) is desired.

If the device, 100 or 200, is operated in the range of dilutions of 4-40, then, given the range of achievable intermediate dilutions of 10-2,000 exemplified in the table above, the portable embodiment of this invention can achieve total (overall) fragrance gas-phase dilutions in the range of 40-80,000. Based on our investigation of fragrance trail with CFD simulations, the system 300 operating at the aforementioned range of dilutions can reproduce real-life fragrance trail from a person wearing fragrance corresponding to fragrance deposition areas between 80 cm² and 400 cm² on the body (skin, clothes, and/or hair) of the wearer at the distances between 0.5 meters and 9 meters from the fragrance wearer, although the utility of the device is not limited by the aforementioned set of conditions.

Similar operational considerations apply for a non-portable embodiment of this system 300 that utilizes the 10-L device, 100 or 200. Given the range of dilutions achievable, the system 300 in its various embodiments can reproduce consumer usage of air fresheners and other consumer products in spaces from 2 m³ (e.g., 1 m by 1 m by 2 m) to 100 m³ (6 m by 5.5 m by 3 m), although based on the size and type of the fragranced product, it may be desirable to operate the system 300 outside of the aforementioned range, said system 300 capable of such flexibility.

The table below represents optimal operating conditions for the system 300 object of the present invention:

| | Range/ratio/ | Preferred Range/ratio/ |
|---|---|---|
| Feature | Parameter | Parameter |
| Volume | 20 mL-20 L | 30 mL-10 L |
| of device, | | |
| 100 or 200 | | |
| Surface area of | 0.18 cm²-1,000 cm² | 0.3 cm²-500 cm² |
| fragrance | | |
| release on | | |
| the fragrance | | |
| source support | | |
| Air flow rate | 20 mL/min-10 L/min | 40 ml/min-7 L/min |
| in device, | | |
| 100 or 200 | | |
| Air flow rate | 0 L/min-100 L/min | 1 L/min-50 L/min |
| in dilution | | |
| stream(s) | | |
| Air flow rate | 0 L/min-99.9% of air | 0 L/min-99.9% of air |
| in waste | flow in dilution | flow in dilution |
| stream(s) | stream(s) | stream(s) |
| Air flow rate | 0.5 L/min-20 L/min | 0.8 L/min-10 L/min |
| at smelling | | |
| port(s) | | |
| Temperature | 20° C.-35° C. | 22° C.-32° C. |
| in device, | | |
| 100 or 200 | | |
| Number of | 1-2 | 1-2 |
| auxiliary | | |
| air dilution | | |
| streams | | |
| Number of | 1-2 | 1-2 |
| waste air | | |
| streams | | |

The command means 320 is, for example, a computer program configured to emit control commands (activation, deactivation and associated operational parameters) to mass flow controllers, pumps and/or compressed air sources.

In particular embodiments, such as represented in FIG. 5, the command unit 310 further comprises a scent trail simulation means 325, activation commands being transmitted by the command means 320 as a function of parameters of the simulated scent trail.

Said trail simulation means 325 is, for example, a computer software configured to associate a dilution ratio to a value of distance. Said dilution ratio can be linearly or non-linearly related to a value of distance. In one such embodiment, the dilution ratio can be multiplied by a factor of ten for each meter from the fragrance source.

In such embodiments, the value of distance can be predetermined or set, via a computer interface, by a user. Depending on the value of distance considered, the trail simulation means 325 calculates the dilution ratio, and that dilution ratio is then used by the command means 320.

In particular embodiments, such as represented in FIG. 5, the system 300 comprises a virtual reality headset 330 or an electronic display (not represented), the dilution and/or waste ratio being determined as a function of an execution parameter of said virtual reality headset 330 or electronic display.

A virtual reality headset 330 can be of any type known to a person skilled in the art, such as the Occulus Rift (Trademarked) or the HTC Vive (Trademarked). Substantially, a virtual reality headset 330 is an electronic display configured to be mounted upon the head of a user to immerse said user into a virtual environment, as a means to enhance the experience of the user.

An electronic display is, for example, a computer screen configured to display a user interface preferably allowing for user interaction.

An example of an execution parameter of either virtual reality headset 330 or electronic display is a virtual distance separating the user of said virtual reality headset 330 or electronic display from a virtual fragrance source located within the virtual environment. Such execution parameter can be provided as a value of distance to a trail simulation means 325 as described above.

In particular embodiments, the dilution ratio decreases or increases over time.

Such embodiments allow for the sensory reproduction of the user approaching or moving away from a fragrance source.

In embodiments comprising an elbow 435, said elbow 435 can be located between a first set of inlet 425 and outlet 431 and a second set of inlet 426 and outlet 430.

The elbow 435 can further be positioned between any two said elements. For example, the elbow 435 can be positioned between the inlet 425 and the outlet 431.

In particular embodiments, such as represented in FIG. 3, the conduit 420 comprises a needle 440 or any other geometric obstacle in the air flow path, such as protruding rings or fins, configured to increase mixing within the conduit 420. In more general terms, a needle is one example of an object with a specific geometry that partially obstructs the flow through the conduit 420, with the objective of increasing air mixing within said conduit.

The needle 440 can be formed of any material. Preferably, the selected material is configured to not absorb scent from or release scent into the air flow said needle 440 is designed to agitate. The needle 440 can be positioned longitudinally along the conduit 420 or transversally.

In particular embodiments, such as represented in FIGS. 3 and 5, the system 300 further comprises a heating apparatus 445, said heating apparatus 445 being configured to change temperature of the air flow pathways from the inlet 405 to the outlet 410. In variants, the heating apparatus 445 is also configured to heat the device, 100 or 200. In variants, the heating apparatus 445 is configured to heat only part of the system 300.

Such a heating apparatus 445 is, for example, resistive (Ohmic) and powered by an electric energy source, part of the system 300 or external to said system 300. Preferably, said heating apparatus 445 is activated and de-activated by a command system, such as a microcontroller.

In particular embodiments, the system 300 comprises a charcoal filter or other scrubber means connected to the outlet or outlets, 430 and/or 431, for waste scented air.

In particular embodiments, such as represented in FIG. 3, the system 300 further comprises an air flow restrictor 450 upstream of the outlet 410 for mixed scented air.

Such a restrictor 450 can be of any type known to a person skilled in the art. Said restrictor 450 is configured to limit the flow rate of air presented to the user via the smelling port(s) as well as to increase the air pressure in the system 300, upstream of said restrictor 450.

FIG. 3 also shows a particular embodiment of the assembly object of the present invention. This assembly to form a system 300 according to any FIGS. 3 to 5, comprises:
a compact scent delivery device 200 such as disclosed in regard to FIG. 1 or 2,
an air flow mixing unit 400 comprising:
   an inlet 405 for scented air, configured to be connected to the outlet 125 of scented air of the compact scent delivery device,
   an outlet 410 for mixed scented air directed at a smelling port 415,
   a conduit 420 connecting the inlet for scented air and the outlet for mixed scent air and
   at least one element of; and preferably one of each of, the following:
      an inlet 425 for fresh air, configured to be connected to a fresh air delivery apparatus, said inlet delivering fresh air into the scented air stream between in the inlet for scented air and the outlet for mixed scented air and/or
      an outlet 430 for waste scented air, configured to be connected to an air flow retrieval apparatus or a waste air disposal means, said outlet retrieving scented air from the scented air stream between in the inlet for scented air and the outlet for mixed scented air to reduce the air flow.

In particular embodiments, such as represented in FIGS. 3 and 5, the system 300 comprises at least two compact scent delivery devices 200 such as disclosed in regards to either FIG. 1 or 2 and, for each compact scent delivery device 200, an independent mixing unit, 400 and 401, said system further comprising a scented air mixer 335 connected to the outlet 410 for mixed scented air of each said mixing unit, said scented air mixer further comprising an outlet 340 for mixed scented air directed at its own dedicated smelling port.

The scented air mixer 335 can be, for example, a singular chamber connected to the outlets 410 of the delivery devices 200, the connection between outlet 410 and said chamber being regulated by a valve commanding the relative quantity of air flow entering the chamber from each delivery device 200. Such an embodiment allows for the dynamic change of the ratio within the produced mixed air flow.

In particular embodiments, such as represented in FIG. 5, the system 300 comprises:
a mixed scented air ratio determination means 345 and
a command means 350 configured to transmit activation commands to at least one pump or compressed air source as a function of the mixed scented air ratio determined.

The command unit 310 may comprise the mixed scented air ratio determination means 345 and the command means 350, such as shown in FIG. 5.

The mixed scented air ratio determination means 345 is, for example, a computer program configured to run a computing system, such program being activated by a user interface configured to receive user input. In this interface, the user can set the ratio directly, said ratio being used by the command means 350.

The mixed scented air ratio can be determined automatically as a function of values captured by logical or physical sensors. For example, when the system 300 comprises a virtual reality headset, two virtual fragrance sources can be located within the virtual environment, each virtual fragrance source being associated with a particular device 200 and corresponding mixing unit, such as 400 and 401, respectively.

As the user moves within the virtual environment, the position of said user approaches either source or, depending upon the ratio of distances, the ratio of mixing of each corresponding scented air flow can be determined. Other parameters can be used, such as the direction the user is facing within the virtual environment.

The command means 350 is similar to the command means 325.

The system 300 of FIGS. 3 to 5 can be portable or stationary. Such a system 300 may comprise an autonomous electric energy source, such as a battery, powering the electric components of said system 300.

In particular embodiments, the system 300 may be used to provide multi-channel operation with the ability to test several fragrances at once. In such embodiments, the system 300 comprises a plurality of devices, 100 and/or 200, each device, 100 and/or 200, being associated to a dedicated mixing unit 400 and to a dedicated smelling port. In variants, several devices, 100 and/or 200, are associated to a common mixing unit 400, the system 300 comprising a scented air input selector activated automatically or manually. This selector is configured to direct the air flow of only one of the devices, 100 and/or 200, to the mixing unit 400. Several mixing units can be put in parallel and the resulting scented air streams then selectively mixed. Similarly, several devices, 100 and/or 200, could feed into one mixing unit configured to selectively mix the air streams originating from said devices, 100 and/or 200.

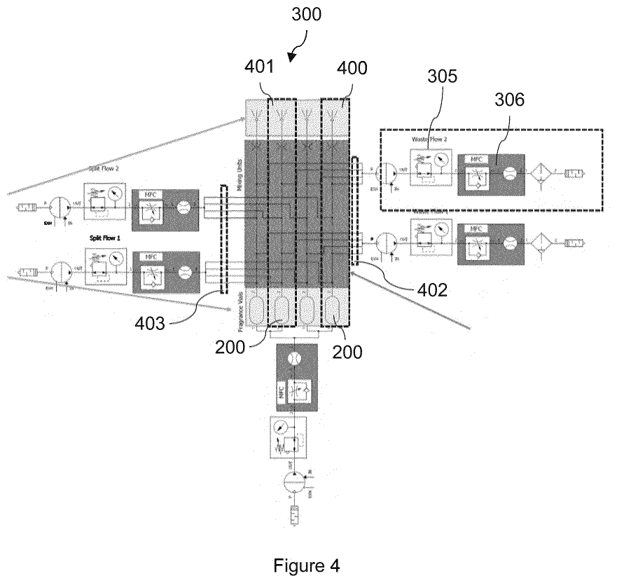

In particular embodiments, such as shown in FIG. 4, the system 300 comprises a network of air flow supply 403 and removal lines 402.

In simple variants, one waste scented air pump can be connected to one or more outlet, 430 and/or 431, of waste scented air of at least one mixing unit, 400 and/or 401. In more complex embodiments, one waste scented air pump is connected to a single outlet, 430 or 431, of waste scented air of at least one mixing unit, 400 and/or 401. In more complex embodiments, such as shown in FIG. 4, the system 300 comprises two waste scented air pumps, each pump being connected to a single outlet, 430 or 431, of waste scented air of four separate mixing units.

In simple variants, one fresh air pump can be connected to one or more inlet(s), 425 and/or 426, of fresh air of at least one mixing unit, 400 and/or 401. In more complex embodiments, one fresh air pump is connected to a single inlet, 425 or 426, of fresh air of at least one mixing unit, 400 and/or 401. In more complex embodiments, such as shown in FIG. 4, the system 300 comprises two fresh air pumps, each pump being connected to a single inlet, 425 or 426, of fresh air of four separate mixing units.

In particular embodiments, components of the system 300 may be subject to chemical passivation treatment.

A typical use case of the system 300 may be:
opening a lid of the device, 100 or 200,
placing a fragrance source (such as a product or substrate containing fragrance) on a height-adjustable support (pedestal) inside the device, 100 or 200,
closing the lid of the device, 100 or 200,
setting a desired air flow rate through the device, 100 or 200,
setting a desired air dilution ratio(s) for subsequent serial dilution(s) of the fragranced air egressing the device, 100 or 200,
setting desired air flow rate for dispensing fragranced air for presentation to evaluator(s) at the smelling port(s),
activating air flow in the system 300,
allowing 5-30 minutes for the system 300 to achieve steady operation (depending on the embodiment and system settings) and
evaluating olfactive performance of the fragrance at the above operating conditions by sniffling egressing fragranced air at the output(s) from the device.

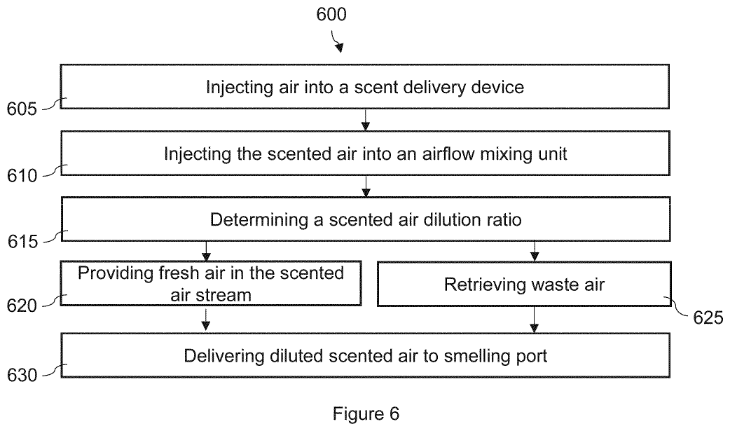

FIG. 6 represents, schematically, a succession of steps of a particular embodiment of the method 600 object of the present invention. This method 600 of scent delivery, comprises:
a step 605 of injecting air into a compact scent delivery device 200 such as disclosed with respect to FIG. 1 or 2,
a step 610 of injecting the scented air, from the outlet of the compact scent delivery device, into an air flow mixing unit 400,
a step 620 of providing fresh air into the scented air stream in the mixing unit and/or a step 625 of retrieving waste scented air from the mixing unit and
a step 630 of delivering properly diluted scented air to smelling port(s) of the system.

In particular embodiments, the method 600 object of the present invention comprises a step 615 of determining a scented air dilution ratio, at least one step of providing 620 and/or retrieving 625 being operated as a function of the ratio determined.

In particular embodiments, the ratio determined during the step 615 of determining is determined as a function of a simulation parameter of distance between a scent source, represented by the scented air at the outlet of the scent delivery device, and a location or distance of perception of said scent.

Said distance may correspond to a virtual distance in a simulated environment displayed upon a digital screen, for example.

Examples of embodiments of such steps are disclosed with regards to the corresponding functional description of FIGS. 1 to 5.

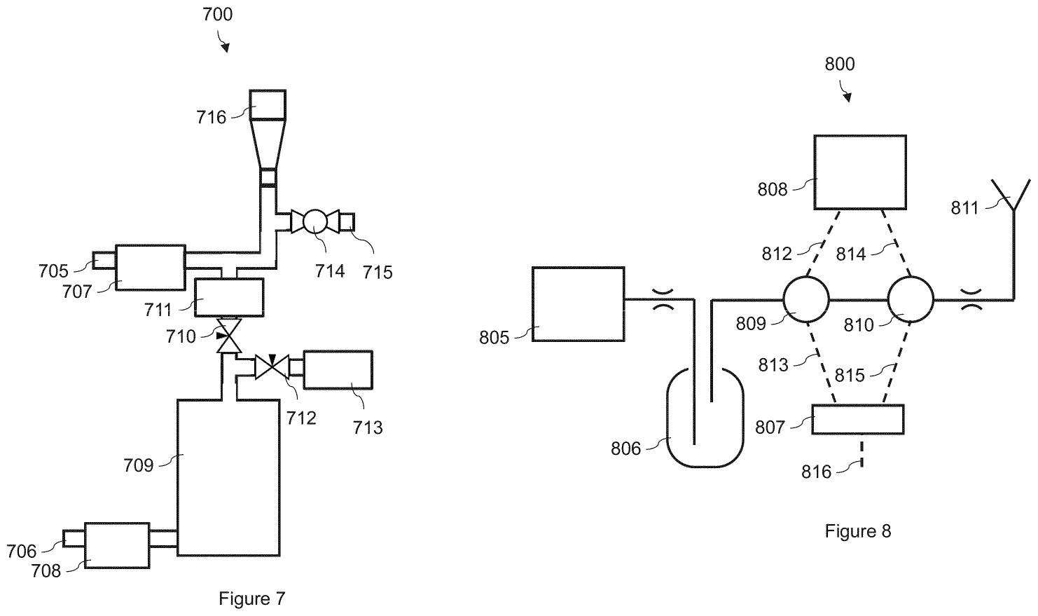

FIG. 7 represents, schematically, a particular, simplified embodiment of the system 700 object of the present invention. This system 700, which is a variant of the system 300 exemplified in FIG. 3, comprises:
a compact scent delivery device 709, which is a variant of the scent delivery device 100 exemplified in FIG. 1,
an inlet 706 for fresh air connected to a compact scent delivery device 709 via a mass flow controller 708 configured to release fresh air from the inlet 706 as a function of the airflow of scented air required by the system 700,
a waste scented air outlet associated with a mass flow meter 713, said mass flow meter 713 being connected to a valve 712,
a scented air outlet, configured to transport air from the compact scent delivery device 709, associated with a valve 710 and a mass flow meter 711,
an air inlet 705 associated with a mass flow controller 707, configured to dilute the stream of scented air let through by the valve 710,
a waste scented air outlet 715 associated with a valve 714 and a mass flow meter (not represented) and
a smelling port 716.

Such a system 700 operates in a way that a quantity (or flow rate) of scented air can be controlled via the mass flow controller 708 and this scented air may be sent to waste prior to dilution, diluted, sent to waste after dilution or sent towards the smelling port after dilution. The specific ratio of each of those alternatives depends on the determined desired concentration of scented air to be smelled by a user at the smelling port, as well as the specifications of the fragrance source (size, type, activation) placed inside the scent delivery device 709.

FIG. 8 represents, schematically, a particular embodiment of the system 800 object of the present invention. This system 800, which is a pressurized variant of the system 300 exemplified in FIG. 3, comprises:
a compressed air source 805, configured to supply a compact scent delivery device 806 continuously with fresh air,
two mixing chambers, 809 and 810, successively positioned at the outlet for scented air of the compact scent delivery device 806, such mixing chambers together comprising a variant of the air flow mixing unit 400 exemplified in FIG. 3 or the air flow mixing unit 900 exemplified in FIG. 9,
a compressed air source 808, associated with a mass flow controller, 812 and 814, associated with each mixing chamber, 809 and 810, respectively,
a waste scented air outlet 816, associated with an optional carbon fiber filter 807 and a mass flow controller, 813 and 815, associated with each mixing chamber, 809 and 810, respectively, and
a smelling port 811 configured to receive the diluted scented air (not sent to waste) for evaluation by a user.

In this variant, any means used in the system disclosed in regard to FIG. 5 may be used as well.

In this variant, the system 800 as a whole is pressurized to create a pressure gradient capable of driving the flow of scented air and fresh air along the entirety of flow paths present in the system 800, including inlet to the scent delivery device 806, inlets to the mixing chambers 809 and 810, outlet(s) for waste scented air 816, and the smelling port 811. In such variants, the pressurization of system 800 acts in lieu of the inlet pumps and the waste pumps.

In further variants of any system disclosed above, the stream of scented air, before or after dilution, may enter a passive, partially obstructed mixing chamber configured to force the air flow along a path increasing the degree of mixing between the scented air and a flow of fresh air.

As it is understood, in some variants, the system is configured to operate under pressurized conditions. The term “pressurized” means in this instance that all or part of the system operates at pressures above atmospheric (ambient) and that a pressure gradient is established within the device and released at the waste scented air outlet(s) and smelling port(s).

1. Compact scent delivery device (100, 200), characterized in that it comprises:
a dynamic air flow chamber (105), comprising:
an inlet (110) for fresh air,
a fragrance source support (120, 220),
an outlet (125) for scented air and
the fragrance source support, fragrance source support being located within the chamber between the inlet and the outlet along a stream of air traversing the chamber.

2. Compact scent delivery device (100, 200), according to claim 1 in which the chamber (105) comprises:
the inlet (110) for fresh air, said inlet facing a reflection surface (115) configured to disperse and reflect and disperse the air from the inlet towards the fragrance source support (120, 220),
said reflection surface,
the outlet (125) for scented air and
a narrowing air flow guide (130) located between the fragrance source support and the outlet along a stream of air traversing the chamber.

3. Device (100, 200) according to claim 2, in which the fragrance source support (120, 220) comprises an air flow guide (135).

4. Device (100, 200) according to claim 3, in which the fragrance source support (120, 220) comprises a perforated surface (121, 221), perforations of the surface forming the air flow guide (135).

5. Device (100) according to claim 4, in which the perforated surface (121) spans the cross-section of the chamber (105).

6. Device (100) according to claim 4, which comprises a perforation adaptation means (135) configured to close/open at least one perforation of the surface.

7. Device (100, 200) according to claim 2, in which an opening of the inlet (110) located within the chamber (105) is located at a distance from the reflection surface (115) less than or equal to three times the width of said opening.

8. Device (200) according to claim 1, in which the inlet (110) extends longitudinally within the chamber (105), said inlet comprising a fixture (240) for the fragrance source support.

9. Compact scent delivery system (300), characterized in that it comprises:
a compact scent delivery device (200) according to claim 1,
an air flow mixing unit (400) comprising:
an inlet (405) for scented air, configured to be connected to the outlet (125) of scented air of the compact scent delivery device,
an outlet (410) for mixed scented air directed at a smelling port (415),
a conduit (420) connecting the inlet for scented air and the outlet for mixed scent air and
at least one element of:
an inlet (425) for fresh air, configured to be connected to a fresh air delivery apparatus, said inlet delivering fresh air into the scented air stream between in the inlet for scented air and the outlet for mixed scented air and/or
an outlet (430) for waste scented air, configured to be connected to an air flow retrieval apparatus and/or a waste air exhaust, said outlet retrieving scented air from the scented air stream between the inlet for scented air and the outlet for mixed scented air in order to reduce the air flow either for subsequent dilution and/or for the associated smelling port (415).

10. System (300) according to claim 9, in which the conduit (420) comprises at least one elbow (435) or flow modification means.

11. System (300) according to claim 10, which further comprises at least two elements among:
an inlet (425, 426) for fresh air, configured to be connected to a fresh air delivery apparatus, said inlet delivering fresh air into the scented air stream between the inlet for scented air and the outlet for mixed scented air and/or
an outlet (430, 431) for waste scented air, configured to be connected to an air flow retrieval apparatus and/or a waste air exhaust, said outlet retrieving scented air from the scented air stream between the inlet for scented air from the compact scent delivery device and the outlet for mixed scented air to the smelling/evaluation port(s), in which the elbow (435) or flow modification means is located between two said elements.

12. System (300) according to claim 9, in which the conduit (420) comprises a needle (440) or any other geometric obstacle in the air flow configured to increase mixing within the conduit.

13. System (300) according to claim 9, in which at least one inlet (425) for fresh air or outlet (430) for waste scented air is oriented perpendicularly to the air stream with which at least one said inlet (425) for fresh air or outlet (430) for waste scented air interacts.

14. System (300) according to claim 9, which further comprises a heating apparatus (445), said heating apparatus being configured to air flow from the inlet (405) to the outlet (410).

15. System (300) according to claim 9, which further comprises an air flow restrictor (450) upstream of the outlet (410) for mixed scented air.

16. System (300) according to claim 9, which further comprises one of the following:
at least one pump (305) connected to an inlet (110, 425, 426) for fresh air or an outlet (430, 431) for wasted scented air, or compressed air source (805, 808) connected to an inlet (110, 425, 426) for fresh air.

17. System (300) according to claim 16, which further comprises a command unit (310) comprising:
a dilution and/or waste ratio determination means (315),
at least one mass flow controller (306) connecting at least one:
said pump (305) to an inlet (110, 425, 426) for fresh air or an outlet (430, 431) for wasted scented air, or
a compressed air source (805, 808) to an inlet (110, 425, 426) for fresh air, or
an outlet (430, 431) to a waste scented air disposal means (816) and
a command means (320) configured to transmit activation commands to at least one said mass flow controller as a function of the dilution and/or waste ratio determined.

18. System (300) according to claim 17, in which the command unit (310) further comprises a scent trail simulation means (325), activation commands being transmitted by the command means (320) as a function of parameters of the simulated scent trail.

19. System (300) according to claim 17, which comprises a virtual reality headset (330) or an electronic display, the dilution and/or waste ratio being determined as a function of an execution parameter of said virtual reality headset or electronic display.

20. System (300) according to claim 17, in which the dilution ratio decreases or increases over time.

21. System (300) according to claim 9, in which a compact scent delivery device (200) is positioned so that the scented air outlet (125) is higher along the upward vertical direction than the inlet (110) for fresh air of said device.

22. System (300) according to claim 9, which comprises a moisture trapping means (355) and/or an activated carbon trapping means (356) positioned along the stream of air traversing the system.

23. System (300) according to claim 9, in which components of the system 300 subjected to scented air are chemically passivated.

24. System (300, 700, 800) according to claim 9, configured to operate under pressurized, above atmospheric pressure, conditions.

25. Assembly (500) to form a system according to claim 9, characterized in that it comprises:
a compact scent delivery device (100, 200) characterized in that it comprises:
a dynamic air flow chamber (105), comprising:
an inlet (110) for fresh air,
a fragrance source support (120, 220),
an outlet (125) for scented air and
the fragrance source support, fragrance source support being located within the chamber between the inlet and the outlet along a stream of air traversing the chamber;
an air flow mixing unit (400) comprising:
an inlet (405) for scented air, configured to be connected to the outlet (125) of scented air of the compact scent delivery device,
an outlet (410) for mixed scented air directed at a smelling port (415),
a conduit (420) connecting the inlet for scented air and the outlet for mixed scent air and
at least one element of:
an inlet (425) for fresh air, configured to be connected to a fresh air delivery apparatus, said inlet delivering fresh air into the scented air stream between in the inlet for scented air and the outlet for mixed scented air and/or
an outlet (430) for waste scented air, configured to be connected to an air flow retrieval apparatus and/or a waste air exhaust, said outlet retrieving scented air from the scented air stream between the inlet for scented air and the outlet for mixed scented air in order to reduce the air flow either for subsequent dilution and/or for the associated smelling port (415).

26. Method (600) of scent delivery, characterized in that it comprises:
a step (605) of injecting fresh air into a compact scent delivery device (100, 200) according to claim 1,
a step (610) of injecting the scented air, from the outlet of the compact scent delivery device, into an air flow mixing unit (400) and
a step (620) of providing fresh air into the scented air stream in the mixing unit and/or a step (625) of retrieving waste scented air from the mixing unit.

27. Method (600) according to claim 26, which comprises a step (615) of determining a scented air dilution ratio, at least one step of providing (620) and/or retrieving (625) being operated as a function of the ratio determined.

28. Method (600) according to claim 27, in which the ratio determined during the step (615) of determining is determined as a function of a simulation parameter of distance between a scent source, and a location of perception of said scent, said distance influencing intensity, perceivability and/or recognizability of the scented air at the outlet of the scent delivery device.
